(19) United States
(12) Patent Application Publication
(10) Pub. No.: US 2014/0271512 A1
(43) Pub. Date: Sep. 18, 2014

(54) COMBINATION DERMATOLOGICAL COMPOSITION AND USE

(71) Applicant: Loretta Ciraldo, Davie, FL (US)

(72) Inventor: Loretta Ciraldo, Davie, FL (US)

(21) Appl. No.: 14/215,410

(22) Filed: Mar. 17, 2014

Related U.S. Application Data
(60) Provisional application No. 61/787,468, filed on Mar. 15, 2013.

(51) Int. Cl. A61K 8/68; B65D 25/38; A61Q 19/08; B65D 85/00; A61K 8/365; A61K 8/55

(52) U.S. Cl. CPC A61K 8/68; A61K 8/365; A61K 8/553; A61Q 19/08; B65D 85/70; B65D 25/38
USPC 424/63; 514/25; 206/232; 206/229

(57) ABSTRACT

Compositions and methods for enhancing the aesthetic appearance of the skin of an animal, including the visible reduction of lines, creases, or wrinkles of the face, are achieved by applying to the surface of the skin a mixture of an aqueous composition containing urea, lactic acid, calcium pantothenate, and a lipophilic composition containing a lipid such as ceramide, lecithin, or a glycolipid. Optionally, glycerin can be included in the compositions.

### CROSS-REFERENCE TO RELATED APPLICATIONS

This application claims the benefit of U.S. Provisional Application Ser. No. 61/787,468, filed on Mar. 15, 2013, entitled Dermatological Composition and Use, the prior application is herewith incorporated by reference in its entirety.

### BACKGROUND OF THE INVENTION

The skin is the largest organ in the body and serves several important functions that are essential to life. The skin retards dehydration and also acts as a barrier to the invasion of various pathogens and noxious substances.

Skin is composed of the epidermis, or upper layer, and the subjacent dermis. The epidermis gives rise to the nails, hair, sebaceous glands, sweat glands, and the parenchyma of mammary glands. The epidermis is composed of several layers, including the outermost layer known as the stratum corneum, which is comprised of epidermal cells embedded in an intercellular matrix of semi-polar and polar lipids. This lipid layer of the stratum corneum assists in the retention of water. The water content of the stratum corneum has a profound influence on the appearance, flexibility, texture, and dryness of the skin, and also on the absorption of drugs and other molecules into and through the skin.

With increasing age, the skin gradually becomes drier, less flexible and less supple, more wrinkled, and can appear scaly in appearance. One of the major objectives of the cosmetic industry is to retard the drying and wrinkling of the skin that occurs with normal aging or as the result of exposure to wind, sun, cold and chemicals. Increasing the moisture content of the skin is one mechanism for achieving this objective.

Skin moisturizing or protective compositions, which include one or more of the ingredients used in the subject composition, have been described. For example,

U.S. Pat. No. 4,146,649 describes a skin moisturizing composition composed of polyethoxylated fatty alcohol, polyethoxylated glycoside, water and minor quantities of water soluble salt and amino acid or urea, which penetrates the skin surface and retains moisture near the skin surface.

U.S. Pat. No. 5,945,409 describes methods and compositions requiring lecithins for topically treating and moisturizing keratinous structures of humans and animals including skin.

U.S. Pat. No. 5,954,836 describes compositions and methods for inhibiting skin irritation by the application of an anti-irritant amount of aqueous-soluble divalent calcium cation. The '836 patent does not describe a formulation of the subject invention.

U.S. Pat. No. 6,316,428 describes methods and compositions comprising lecithin and a poloxamer for topically treating and moisturizing keratinous structures of humans and animals including skin.

U.S. Pat. No. 6,423,323 describes a fatty-acid containing foam skin cream obtainable by a process which includes melting at 75 C.

U.S. Pat. Nos. 7,763,595 and 7,928,090 describe skin moisturizing methods and compositions comprising an acyl glucosamine derivative.

U.S. Pat. No. 7,816,310 describes a skin moisturizing composition having at least one hydroxyalkylurea (HAU) in combination with at least one other moisturizing agent.

U.S. Pat. No. 7,910,090 describes a non-irritating, photoprotective cosmetic base composition which includes one or more of electrolyte, buffer, mild preservative, lubricant, or any combinations thereof.

Although certain of the ingredients of the subject composition have been used in topical skin treatments, as described in the above patents, the specific compositions and combinations according to the subject invention have not, to our knowledge, been heretofore described. Moreover, none of the previously described compositions are known to exhibit the exceptional, and advantageous, skin rejuvenation results exhibited by the subject invention.

### SUMMARY OF THE INVENTION

The present invention is generally related to a topical dermatologic composition and method for moisturizing and rejuvenating keratinous tissues including skin, hair, fingernails, and toenails of humans and animals, and also hooves and horns of animals. Specifically, the present invention relates to a topical composition, referred to in the cosmetic industry as a “topical filler,” used or formulated in combination with a skin protective agent, and methods for treating and reducing the appearance of facial lines, creases, or wrinkles of a subject due to aging or environmental factors.

More particularly, the subject invention concerns a combination of a first aqueous composition and a second lipophilic composition, as well as a method combining these compositions for enhancing or improving the appearance of lined or wrinkled skin, without use of an injection or plastic surgery.

A first preferred composition of the subject invention comprises urea, lactic acid or lactate (typically as the magnesium or potassium salt), and calcium pantothenate, and a second preferred composition comprises one or more lipid, such as sphingolipid (e.g. ceramide), phospholipid (e.g., lecithin), or a glycolipid, wherein the first and second compositions, when used together, can provide unexpected enhancement or improvement to the appearance of facial and body skin susceptible to aging, lines or creases, wrinkles, dry appearance, and the like. The first and second compositions of the invention can further comprise glycerin or a glyceryl component.

The present invention includes a method for moisturizing keratinous tissue of a human or animal comprising the step of topically applying to the keratinous tissue of the human or animal, a mixture of the first composition comprising urea, lactic acid, calcium pantothenate, and the second composition comprising a lipid, preferably one or more of ceramide, lecithin, or glycolipid, whereby the intracellular and intercellular components of the stratum corneum, epidermis and dermis can be replaced to restore youthful and healthful appearance to the skin. Another embodiment of the present invention comprises a method of treating keratinous tissue of a human or animal comprising the step of topically applying to the keratinous tissue of the human or animal, a mixture of the first composition comprising urea, lactic acid, calcium pantothenate, and the second composition comprising one or more lipids, such as a sphingolipid, phospholipid, or glycolipid e.g., ceramide, lecithin, or the like.

In one embodiment, the present invention provides an easy-to-use therapeutic and cosmetic process for treating and moisturizing skin, especially the face and hands, and reducing the appearance of wrinkles, lines or creases, or dry skin by topically applying the composition disclosed herein. Use of this process and composition increases the moisture content of intracellular and extracellular components of the skin to reduce wrinkling, and provide a rejuvenated appearance to the skin.

In addition, this invention can have utility for the treatment of dry and brittle fingernails and toenails, and the adjacent skin that often is dry and cracked.

In another embodiment, this invention is a therapeutic and cosmetic process for application to animals with cracked and damaged horns and/or hooves. A horn or hoof dressing in show horses and cattle would produce a more vibrant and healthy appearance. In addition, rapid closure of cracks in hooves would prevent invasion by foreign material and can reduce the incidence of infection.

An additional embodiment of the invention is a therapeutic and cosmetic process for the treatment of dry and thinning hair to provide increased hair growth and thickness.

The compositions of the present invention can include other pharmaceutically acceptable components such as gelling agents, compounding agents, scents or fragrances, coloring agents, and the like. The composition of the present invention can also include other pharmaceutically active agents such as antibacterial, antifungal, antiprotozoal or antiviral agents.

This invention addresses the need for an easy-to-use topical treatment for cosmetic and therapeutic purposes of keratinous structures including the skin, nails, and hair of humans and animals, and also for the treatment of the nails, hair, hooves, and horns of animals.

Accordingly, it is an object of the invention to provide a composition and method for treating skin of humans and animals, to increase the moisture of the skin, and thereby reduce the appearance of lines, creases, wrinkles, or dryness which can occur from environmental exposure or aging.

It is another object of the invention to provide a composition and method for treating skin of humans and animals to decrease the wrinkled appearance of the skin.

Still another object of the invention is to provide a composition and method for treating skin to rejuvenate the skin and decrease the aged appearance of the skin.

It is another object of the invention to provide a method for topically applying an improved composition to animal or human keratinous tissue for enhancing the penetration of pharmacologically active substances into keratinous tissue, especially the epidermis and dermis of the skin, without damaging the tissue or causing adverse systemic effects.

Still another object of the invention is to provide a composition and method for treating windburn, chapped lips, sunburn, and dehydrated skin due to exposure to chemicals.

Still another object of the invention is to provide a composition and method for treating calluses, corns, and any other skin conditions involving drying of the skin.

These and other objects, features and advantages of the present invention will become apparent after a review of the following detailed description of the disclosed embodiments and the appended claims.

### DETAILED DESCRIPTION OF THE INVENTION

The present invention is a method of moisturizing keratinous tissue of a human or animal comprising the step of topically applying to the keratinous tissue of the human or animal, a first aqueous composition comprising urea, lactic acid or lactate salt, calcium pantothenate, and a second lipophilic composition comprising one or more lipids, e.g., ceramide, lecithin, glycolipid, or the like, whereby the compositions or combination thereof are delivered into the stratum corneum, epidermis or dermis to replace both intracellular and extracellular components of the skin, thereby enhancing the appearance, texture, or feel of the skin.

The first aqueous composition can further include glycerin. The second lipophilic composition can further include a glyceryl compound.

Another embodiment of the present invention comprises a method of treating keratinous tissue of a human or animal comprising the step of topically applying to the keratinous tissue of the human or animal, a first composition comprising urea, lactic acid, calcium pantothenate, and a second composition comprising one or more lipid components, such as lecithin, ceramide, or one or more glycolipids.

The terms “about” or “approximately” mean within an acceptable range for the particular parameter specified as determined by one of ordinary skill in the art, which will depend, in part, on how the value is measured or determined, e.g. the limitations of the measurement system. For example, “about” can mean a range of up to 10% of a given value.

As used herein, a “butter” or “botanical butter” is a fat and/or oil extract of a plant fruit and/or seed characterized by having emollient properties and a melting point near human body temperature.

“Conventional wax” or “conventional waxes” include petroleum derived waxes, beeswax, carnauba wax and candelilla wax. These waxes may also be referred to herein as “non-jojoba wax” or “non-jojoba waxes”. Jojoba waxes are distinguishable from conventional waxes in that they are derived from the jojoba plant and they have properties that contribute to structure as well as providing substantial emollient and moisturizing properties not characteristic of conventional waxes.

An “emollient” is a cosmetic ingredient which can help maintain the soft, smooth and pliable appearance of skin tissue. Typically, emollients impart sensory properties of smoothness and/or softness when the composition is applied to the lips.

The “enhanced penetration” caused by compositions of this invention as used in topical application with this method, means increased penetration into the skin, and is achieved with compounds such as lecithin organogel, poloxamer organogel, phospholipid gels or poloxamer phospholipid gels including but not limited to phosphatidylethanolamine, phosphatidylserine, phosphatidylinositol, and phosphatidic acid and phosphatidylcholine optionally combined with n-decylmethyl sulfoxide (NDMS), PLURONIC F127, ethoxy diglycol, ethanol, or cholesterol. Enhanced penetration can be observed in many ways known to those skilled in the art.

The term “keratinous tissue” as used herein, means skin, fingernails, toenails, hooves, horns and hair, and any other cells containing keratin. The present invention provides a method and composition for topically treating damaged or diseased keratinous tissue resulting from any number of causes including, but not limited to: aging; chemical drying; radiation; burns from wind, sun, fire, cold, frostbite, radiation or chemicals; and dehydration resulting from skin disease, abrasion, sun, chemicals, wind, cold, fire, renal disease, colonic disease, hemorrhage, vasopressin imbalances, hypothalamic dysfunction, neurohypophyseal dysfunction, or other endocrine abnormalities.

A “lip balm” is a semisolid composition for application to the lips that has protective and/or moisturizing properties. Lip balms may contain medicaments and/or ingredients that promote lip health, such as sunscreens. Lip balms may also include colorants that may or may not impart the color to the lips. A “stick lip balm” is a lip balm formed into a stick shape that is typically dispensed from a container that permits extension and retraction of the lip balm stick from and into the container.

To obtain emollient properties and desired structure, a mixture of jojoba esters may be used. In view of commercially available jojoba ester products such as Floraesters supplied by FloraTech Americas, 1151 North Fiesta Blvd., Gilbert Ariz. 85233, it may be convenient to use a combination of jojoba ester products.

The term “moisturize” or derivatives thereof, relates to the conservation or enhancement of the water content of the keratinous tissue of animals and humans, with particular reference to the skin, hair, nails, hooves and horns.

“Percent” or “%” as used herein refers to the percentage by weight of the total composition, unless otherwise specified.

The term “pharmacologically active agent” relates to any chemical material or compound suitable for topical administration which includes any desired local effect on animal or human tissue contacted therewith.

The term “pharmaceutically effective carrier” is used herein to mean any liquid, gel, salve, solvent, liquid, diluent, fluid ointment base, liposome, micelle, giant micelle, and the like, which is suitable for use in contact with living animal or human tissue without causing adverse physiological responses, and which does not interact with the other components of the composition in a deleterious manner.

By the term “therapeutically effective amount” of a molecule, drug or pharmacologically active agent is meant a nontoxic but sufficient amount to provide the desired therapeutic effect.

“Topical” application is used to mean local administration of the composition and its various embodiments, for example, in the treatment of dry skin.

The present invention includes a composition or a combination of compositions for topical treatment of keratinous tissue comprising the following components; urea, lactic acid, calcium pantothenate, and one or more lipids, such as a sphingolipid, phospholipid, or glycolipid, e.g., ceramide, lecithin, or the like. The present invention optionally can include glycerin or a glyceryl component.

Solvents used in the preparation of a variety of topical compositions, including gels, all of which are appropriate in practicing the present invention, are described in Scartazzini, et al. Journal of Physical Chemistry 92:829-833, 1988, and Luisi, P. L. et al. Colloid and Polymer Science 268:356-374, 1990, both of which are incorporated herein by reference in their entirety. Specifically these solvents include the following: ethyl laurate, butyl laurate, ethyl myristate, isopropyl myristate, isopropyl palmitate, isooctane, cyclooctane, cyclododecane, methyl cyclohexane, tert-butylcyclohexane, phenylcyclohexane, bicyclohexyl, 1,3,5-triisopropylbenzene, octylbenzene, trans-decalin, (1R)-(+)-trans-pinane, (1R)-(+)-cis-pinane, n-pentane, n-hexane, n-heptane, n-octane, n-nonane, n-decane, n-undecane, n-dodecane, n-tridecane, n-tetradecane, n-pentadecane, n-hexadecane, n-heptadecane, 2,3-dimethylbutene, 1-hexene, 1,7-octadiene, tripropyllamine, tributylamine, triisobutylamine, trioctylamine, dibutyl ether and 2-dodecen-1-yl succinic anhydride.

In addition to isopropyl palmitate and isopropyl myristate, other solvents may be used in the practice of the present invention. These solvents include, but are not limited to the following: mineral spirits, kerosene, isooctane, petroleum ether, diethyl ether, benzene, toluene, methanol, ethanol, heptanol, methyl isobutyl ketone, cyclohexanone, methylene chloride, choloroform, chlorodifluoromethane, tetrahydrofuran, oleyoleate, 2-octyldodecanol, cetyl and stearyl 2-ethylhexanoate, n-octanol, ethyl laurate, isooctane, cyclopentane, cyclohexane, and cycloheptane.

In a preferred embodiment, the first and second compositions are prepared by combining the ingredients to provide a substantially homogeneous mixture in a cream, lotion, or gel form. In a most preferred embodiment, the first composition and second composition are separately prepared and individually placed into respective containers, e.g., dispensing bottles or tubes, for being separately dispensed and combined or mixed by the user prior to use. Preferably, the separate containers can be provided as a single article of manufacture, being contained within a single outer packaging, and further comprising written instructions for dispensing, mixing, and using the first and second compositions.

It is also contemplated that the first composition and second composition can be combined or mixed as a single composition, either prepared separately and mixed together and placed into a single dispensing container, such as a bottle or tube, or where the ingredients of the first and second compositions are prepared or mixed together as a single homogeneous composition which can be placed into a single dispensing container, such as a bottle or tube, whereby the composition can be dispensed as the mixture prior to use.

A further embodiment can comprise a single container housing dual chambers, separately or respectively containing the first aqueous composition and second lipophilic composition. Each chamber can have an individual port or dispenser means (e.g., a pump nozzle) associated therewith for dispensing a predetermined or pre-measured amount of the composition from within its respective chamber. A preferred embodiment of a dual-chamber container can provide an adjustment means associated with one or both dispensers for adjusting the predetermined or pre-measured amount of composition dispensed. Therefore, if a greater or lesser amount of one composition or the other is desired, the adjustment means can be set to dispense the desired amount. For example, for treatment of normal skin, the adjustment means for each dispenser can be set to dispense the same amount of each composition, i.e., a 1:1 ratio of the first and second compositions.

For drier or more damaged or wrinkled skin, or for more oily skin, the adjustment means can be set to dispense a greater or lesser amount of the first aqueous composition, whereby the ratio of the first and second compositions is less than 1:1 or greater than 1:1. Preferably the ratio can be adjusted to dispense the first and second compositions at ratios ranging from about 5:1 to about 1:5.

Alternatively, the separate chambers can communicate with a single dispenser nozzle that can allow for mixture or combination of the compositions at the time the first and second components are dispensed. One preferred embodiment includes a single dispenser nozzle comprising a septum or partition forming a single, separated opening which allows for dispensing the first and second composition separately, but simultaneously or concomitantly from the separated opening of the dispenser nozzle. The user then receives both compositions, which can be mixed together for use. For example, the two separately dispensed compositions can be received into the palm of the hand using a single pump action from a pump dispenser, mixed by rubbing the hands together, and applied to the skin as a mixture.

In a preferred embodiment of the first aqueous composition or preparation, the primary active ingredients are humectants, and preferably includes urea, lactic acid or lactate salt, and calcium pantothenate. The urea was originally provided in amounts ranging from about 0.1% to about 1.0%, more preferably about 0.3% to about 0.5%, and most preferably about 0.4%.

More recently, it has been discovered that urea can more preferably be provided in amounts ranging from about 0.01% to about 1.0%, preferably about 0.05% to about 0.50%. Preferred formulations are exemplified herein having urea provided as little as 0.07% of the composition and up to about 0.40% of the composition.

Lactic acid or lactate salt is provided in amounts ranging from about 0.01% to about 1.0%, more preferably about 0.02% to about 0.7%, and most preferably from about 0.09% to about 0.6%. More preferably, the lactate component is provided as magnesium lactate, from about 0.06% to about 0.5%, magnesium lactate, and most preferably about 0.063% up to about 0.36% magnesium lactate, and as potassium lactate, from about 0.02% to about 0.3% potassium lactate, and most preferably about 0.025 to about 0.24% potassium lactate. Calcium pantothenate is provided in amounts ranging from about 0.1% to about 1.5%, more preferably about 0.1% to about 0.8%, and most preferably about 0.12% to about 0.72%.

Glycerin, also a humectant, can also be used in the first aqueous composition. Glycerin also has lubricating properties which can provide smoothness to the preparation, which can be advantageous for a pleasant feel to the skin, and to facilitate its application. Glycerin is preferably used in forms selected from glycerin, itself, or ethylhexylglycerin. The glycerin and ethylhexylglycerin components are provided from 0.1% to about 20% of the final composition.

More preferably, glycerin is provided in amounts ranging from about 5% to about 25%, and most preferably at about 10% to about 20%, and most preferably between about 15% and about 18% of the final composition.

Ethylhexylglycerin is preferably provided in amounts ranging from about 0.01% to about 1.0%, preferably from about 0.04% to about 0.6%, more preferably about 0.05% to about 0.6%, and most preferably about 0.049% to about 0.58%.

The first aqueous composition can also preferably be formulated with gelling agents or thickeners, such as gums, e.g., xanthan gum, carrageenan, or the like (typically about 1.0% to about 2.0% and preferably about 1.7%); sugars or sugar alcohols, e.g., glucose, caprylyl glycol, or the like (typically about 0.5% to about 2.0% and preferably at least about 1.5%); salts, such as potassium chloride, magnesium chloride, or sodium chloride, or the like (typically about 0.01% to about 0.3%, and preferably about 0.035); preservatives, such as sodium citrate, citric acid, or the like (typically about 0.001 to about 0.5%, and preferably about 0.005%; and solvents or co-solvents, such as ethanol, phenoxyethanol, and water, where water is typically about 50% to about 90%, preferably about 70% to about 85% and the alcohols are about 0.1% to about 1.0%, preferably about 0.6% of the final composition.

The second lipophilic composition of the invention comprises a lipid component, such as a sphingolipid, e.g., ceramide, a phospholipid, such as lecithin, or one or more glycolipids, which is preferably provided in amounts ranging from about 0.001% to about 0.05%. More preferably, ceramide, when used, is provided in amounts ranging from about 0.02% to about 0.05%, and most preferably about 0.029%. Lecithin, when used, is preferably provided between about 0.0005% and about 0.001%, and in one preferred embodiment at about 0.0009%. Glycolipids, can be provided from about 0.0005% and about 0.001%, and in one preferred embodiment at about 0.0009%.

Typically, the composition comprises one or more glycolipids from about 0.0005% to about 0.005%, and further comprises either ceramide or lecithin, preferably within the ranges as described herein.

Ceramide is a sphingolipid, produced naturally by the body and is present as a protective barrier in skin cell membranes. Alternatively, a phospholipid, e.g., lecithin can be used in corresponding amounts, but the use of ceramide has provided advantageous results.

The second lipophilic composition can include additional oils, lipids, and the like; lubricants; vitamins or nutrients (e.g., antioxidants, such as Vitamin A, E, F, or the like); and solvents. For example, a preferred embodiment can comprise ethylhexyl cocoate (about 15% to about 25%, and preferably about 18%; dimethiconol (about 5% to about 20%, and preferably about 14%); glyceryl linoleate (about 0.01% to about 1.5%, preferably about 0.03%); glyceryl linolenate (typically about 0.01% to about 0.5%, preferably about 0.03%); tocopheryl acetate (typically about 0.001% to about 0.5%, preferably about 0.009%; lecithin (typically about 0.0001% to about 0.05%, preferably about 0.0009%); glycolipids (typically about 0.0005% to about 0.005%, preferably about 0.0009%); dimethylmethoxy chromanol (typically about 0.0001% to about 0.05%, preferably about 0.0003%); and phenoxyethanol (typically about 0.1% to about 1.0%, preferably at least about 0.50% and more preferably at least about 0.75%).

Although not intending or wanting to be bound by the following hypothesis, it is believed that the method and composition of the present invention can cause an increase hydration or water content of the skin, perhaps by increasing the fluid content of the epidermis and dermis. It is believed that the composition of the invention enhances diffusion of moisturizers, surfactants, and emollients into and possibly through the epidermal and dermal layers of the skin. Moreover, the subject compositions and methods are believed to replace both intracellular and intercellular components. Specifically, the first aqueous composition can restore the intracellular components of the skin resulting in hydration and “plumping” of the skin cells; and the second lipophilic composition serves as a barrier or “trap,” which can retain the hydration of the skin cells for reduction of lines, creases, wrinkles, and “dryness” of the skin for an extended period of time resulting in longer duration of effect.

Support for this hypothesis is provided in that the first aqueous composition contains components which are naturally present in the skin. The humectant property of the first aqueous composition is known to provide absorption of water up to about 10,000 times the weight of the ingredients in the composition. This absorption of water is believed to act as a “topical filler” and plumping the skin's surface, and has been observed to elicit this effect within minutes of application.

The second lipophilic composition contains the “barrier” lipid molecules, such as ceramide, lecithin, or glycolipids, which can serve as a “trap” for the hydration of the skin cells, providing a desired effect of smoother, younger, or healthier skin for one to several hours.

In accordance with the subject invention, the method of use comprises providing a desired amount of the first aqueous composition, and providing a desired amount of the second lipophilic composition, mixing the compositions together, and applying the mixture to the skin as needed. The desired amounts are amounts needed to cover the surface of the skin at the site or area of application to the body. For example, a user can dispense about 1 ml to about 5 ml of the first composition in the palm of the hand, dispense about 1 ml to about 5 ml of the second composition into the same or other palm, mix the first and second compositions together by rubbing the palms together, and apply the mixture to the area of the body in need of treatment.

The ratio of amounts of the first composition to second composition to be blended together can vary, and may be adjusted according to skin type or condition. For example, a person needing more hydration or less oil may apply the first composition at a higher ratio, such as 2:1, 3:1, 4:1, or 5:1, to the amount of the second composition. Alternatively, hydrated but drier (less oily) skin needing to retain such hydration without additional oil may apply the first composition at a lower ratio, such as 1:2, 1:3, 1:4, or 1:5 to the amount of second composition. These ratios can be adjusted by need or desire based on the current condition of the skin.

The process can be repeated as desired to cover all target skin areas. In one preferred method for optimal results, all target areas of the skin are treated at least once per day, and can be applied two times per day—once in the morning, and once at night before sleep. The method can also include application of the combination more than two times per day, if desired.

It is known in the art that the specific components of the composition can be substituted by other components which have the same or similar properties. For example, exemplary moisturizing oils suitable for use in the composition can include, but are not limited to, sunflower oil, coconut oil, castor oil, vegetable oil, corn oil, aloe vera oil, canola oil, soybean oil, jojoba oil, olive oil, babassu oil, avocado oil, apricot oil, meadowfoam seed oil, macadamia seed oil, oat kernel oil, palm seed oil, safflower oil, sandalwood oil, sesame oil, almond oil, wheat germ oil, cranberry oil and combinations thereof. In view of consumer preferences it is preferable to use oil from a botanical source. Various hydrocarbons, higher fatty acids, fats and oils, esters, higher alcohols, waxes, silicone oils and fluorocarbon oils including squalene, liquid paraffin, vaseline, microcrystalline wax, ozokerite, ceresin, myristic acid, palmitic acid, stearic acid, oleic acid, isostearic acid, cetyl alcohol, hexadecyl alcohol, oleyl alcohol, cetyl-2-ethyl hexanoate, 2-ethylhexyl palmitate, 2-octyldodecyl myristate, 2-octyldodecylgum ester, neopentylglycol-2-ethyl hexanoate, isooctyric acid triglyceride, 2-octyldodecyl oleate, isopropyl myristate, isostearic acid triglyceride, coconut oil fatty acid triglyceride, olive oil, avocado oil, bees wax, myristyl myristate, mink oil, lanolin, dimethylpolysiloxane, ring dimethylpolysiloxane, methylphenylpolysiloxane, silicone resins, polyether modified silicone, amino modified silicone, and the like can be used. Oils may be included in the composition in amounts of about 0.1% to about 90%.

The composition may further comprise one or more antioxidants. Antioxidants may protect the composition from oxidation (e.g. becoming rancid) and/or provide skin conditioning and skin nutritional benefits upon application to the skin. In view of consumer preferences for “natural” products natural antioxidants such as tocopherols are preferred. Tocopherols, tocopheryl acetate, some botanical butters and green tea extracts are exemplary antioxidants suitable for use in the compositions.

Optionally, the composition may further comprise medicaments including, but not limited to, menthol, camphor, eucalyptus, salicylic acid, and derivatives of salicylic acid. Typically medicaments would be added in amounts of less than about 3%. Amounts may vary depending on the potency of the medicament and the matrix in which the medicament is presented.

Optionally, colorant that imparts color to the composition may be included in the composition. The colorant should not be of an amount, particle size, and/or presented in a matrix that would permit transfer of colorant that imparts a color to the lips during application. Natural colorants such as, for example, plant extracts, natural minerals, or carmine are preferred.

Optionally, a sensate may be included in the composition. A sensate is a composition that initiates a sensory perception such as heating or cooling, for example, when contacted with the skin. Exemplary sensates include, but are not limited to, mint extracts, cinnamon extract, and capsaicin. Preferably sensates are derived from natural sources. However, as sensates typically have high potency and accordingly may yield significant impact at low levels, synthetic sensates are within the scope of this invention.

In addition to the components described above, other components which are usually blended into compounded compositions for cosmetic or aesthetic use, including oils, resins, silicone rubber, moisture retaining agents, antioxidants, surface active agents, preservatives, antiinflammatories, ultraviolet light adsorbents, drugs such as vitamins and hormones, sequestering agents, viscosity adjusting agents and perfumes, can be blended into the composition of the present invention.

Compounds that absorb ultraviolet light, and thus serve as a sunscreen can also be include in either composition. Examples of the ultraviolet light absorbent are: the benzoic acid type such as para-amino benzoate (hereafter abbreviated as “PABA”), glyceryl PABA, n-dimethyl PABA butylether and octyldimethyl PABA; the salicylic acid type such as amino salicylate; and the cinnamic acid type such as octyl cinnamate, ethyl-2,4-diisopropyl cinnamate and octylmethoxy cinnamate; the benzophenone type such as 2,4-dihydroxybenzophenone and 2-hydroxy-4-methoxybenzophenone.

It is also possible to apply an emulsification technique by using purified water, water soluble components and appropriate surface active agents to make an oil-in-water type or water-in-oil type emulsified composition such that the water repellency is not lost.

Bases that can be used in compositions for cosmetic or aesthetic use as an example of the skin preparations of the present invention may be selected from a class of bases that are commonly incorporated as cosmetic components and include the following: animal and plant derived materials such as almond oil, avocado oil, olive oil, turtle oil, bees wax, squalene, lanolin and castor oil; mineral materials such as liquid paraffin, ceresin, paraffin wax, microcrystalline wax, vaseline, candelilla wax, carnauba wax and Japan wax; fatty acids such as palmitic acid and stearic acid; alcohols such as ethanol; higher alcohols such as cetanol, stearyl alcohol, lanolin alcohol, octyl dodecanol and polyvinyl alcohol; esters such as isopropyl myristate, butyl stearate, acetoglyceride, acetylated lanolin, glycerin monostearate, polyoxyethylene monoleate ester, polyethylene glycol monostearic acid ester, and ethyl monooleate; amines such as triethanolamine; ethers such as polyoxyethylene cetyl ether; and silicone oils.

Besides these bases, the following components can be used as required: surfactants such as Spans, Tweens, sugar esters and polyhydric alcoholic esters; mucilages such as acacia gum, tragacanth gum, karaya gum, quince gum, pectin, methyl cellulose, hydroxymethyl propyl cellulose and carboxymethyl cellulose sodium; humectants such as glycerin, propylene glycol, sorbitol, diethylene glycol monoethyl ether and citric acid; extracts from various animals and plants; vitamins; amino acids; activators such as agents to enhance blood circulation; various enzymes; antiinflammatories; saccharides; pigments; biocides or preservatives; antioxidants; and perfumes.

The pigments which are employed to provide the coloring to the compounded compositions for cosmetic or aesthetic use are normally provided as dispersions in an oil such as castor oil. The pigment and castor oil are preferably present in the cosmetic of the invention in an aggregate amount of between about one percent and about ten percent. Pigments of this type are standardized in the cosmetic industry and are identified in that industry by FD&C designations, D&C designations, and natural color designations that are compatible with alcohol solutions. The preferred pigments that are employed include D&C Red No. 6, Barium Lake, D&C Red No. 7 Calcium Lake, D&C Orange No. 5, D&C Red No. 27, FD&C Yellow No. 5, FD&C Blue No. 1, Soloron Silver, Timiron Super Violet, and others. The D&C lake colors are all made with aluminum, calcium, barium, potassium, strontium, or zirconium.

Other pigments which may be employed include those of dye or coal tar origin and chemical compounds used as pigments. Inorganic colors such as iron oxides may be utilized. White pigments may be formed of titanium dioxide, zinc oxide, mica, or pearls. Pigments formed of nitro dyes may be utilized if desired. These dyes contain one atom of nitrogen and two of oxygen. However, only a few nitro dyes are certified by the FD&C or D&C because nitro dyes can be absorbed through the skin, some of which can be toxic. However, D&C Yellow No. 5 is one acceptable nitro dye which can be used as a component of the pigment of the invention.

Azo pigments may also be utilized. These pigments are characterized by the presence of the azo bond, and monoazo pigments include the greatest number of pigments within this group.

Another group of suitable pigments is the triphenylmethane group. FD&C Blue No. 1 is the most popular dye of the group and is widely used in the cosmetic industry. Xanthene pigments may also be utilized. This group of pigments includes Berry brilliant, which is widely used in lipstick colors, as well as D&C orange.

Certain of the quinoline dyes are also suitable for use as pigments. There are only two certified cosmetic colors in this category, namely D&C Yellow Nos. 10 and 11. These are bright greenish yellow colors.

The anthraquinone dyes are also suitable for use in formulating the pigment required by the compounded compositions for cosmetic or aesthetic use of the invention. These dyes are widely used in cosmetics because they are not affected by light. D&C Violet No. 2 is one example of such a dye suitable for use in the invention.

Indigo dyes are also suitable for use and have been used in compounded compositions for cosmetic or aesthetic use for many years. D&C Blue No. 6 is one example of a suitable indigo dye.

Pigments of vegetable, animal, or mineral origin may also be utilized according to the invention. One suitable pigment of animal origin is cochineal extract. Natural colors and vegetable compound pigments which may be utilized include alkanet, annatto, carotene, chlorophyll saffron and tumeric, beet juice powder, carmine, alkanet root, caramel, grape skin extract, beta carotene, and henna. Hydroxyascetone and indelible dyes may also be utilized in formulating the pigment in the compounded compositions for cosmetic or aesthetic use of the invention.

In addition, certain chemicals useful as processing aids may be incorporated into the finished product. Titanium dioxide may be used not only to add a white component of color, but also to serve as a sunscreen.

The composition according to the invention thus comprises at least one lipophilic component, which may be chosen, for example, from the usual constituents of a fatty phase such as waxes, oils, gums and/or fatty substances which are pasty, hydrocarbon-containing and/or silicone-containing, and possibly volatile, alone or as a mixture.

Other beneficial agents known to one skilled in the art may likewise optionally be included in the composition. Aloe extracts and natural organic acids are exemplary of other beneficial agents. Natural organic acids including alpha-hydroxy acids may act as exfoliants, for example. Lactic acid is an exemplary alpha-hydroxy acid. Preferably any other beneficial agents are derived from botanical sources.

It will be appreciated that the compounds of the subject invention may be used and administered as the raw compound, itself, or as a physiologically acceptable salt or solvate thereof, or in a composition containing the compound, salt or derivative, isomer, or solvate thereof.

Compositions of the invention may be administered by any suitable route, but topical or transdermal administration is generally preferred.

The present invention increases the water content in the epidermis and/or dermis, enhancing the visco-elasticity of the skin, which decreases the appearance of wrinkles.

### Formulations

A composition of the invention can comprise a dermatologically or cosmetically acceptable carrier to act as a diluent, dispersant or vehicle for the urea, lactic acid, and calcium pantothenate, so as to facilitate their distribution and provide a homogeneous mixture for the first composition, and a homogeneous mixture for the lipid-containing second composition. The compounds may be formulated for application to the hands, feet including soles, face, scalp, neck, trunk, back, limbs, axillae and/or groin of the subject.

Vehicles other than or in addition to water include liquid or solid emollients, solvents, humectants and thickeners. The present invention may be formulated for topical administration in the form of aqueous or non-aqueous solutions, lotions, creams, gels, oils, ointments, foam, mousse, sprays, emulsions, microemulsions, shampoos, soaps and the like.

In one preferred embodiment, the first aqueous composition of the invention is a mono-phase composition, i.e. a composition comprising a single solvent system, such as a gel. Non-limiting examples of formulations are as follows:

### Gels and Solids

Gel compositions can be formulated by simply admixing a suitable thickening agent to the previously described solution or suspension compositions. Examples of suitable thickening agents include, but are not limited to: cross-linked carboxypolymethylene polymers, ethyl cellulose, polyethylene glycols, gum tragacanth, gum karaya, xanthan gums, bentonite and other clays, hydroxyalkyl cellulose, including hydroxyethyl and hydroxypropyl cellulose. The gelled compositions contain an effective concentration of the active agents; from about 5% to about 75% of an organic solvent as previously described; from about 0.5% to about 20% of a thickening agent, and the balance being water, another aqueous carrier or a combination of carriers.

Compositions of solid forms may be formulated as stick-type compositions intended for application to the skin. The solids also contain from about 50% to about 98% of the previously described emollients. This composition can further contain from about 1% to about 20%, of a suitable thickening agent, and, if desired or needed, emulsifiers and water or buffers. Thickening agents previously described with respect to lotions are suitably employed in the compositions in solid form.

Other ingredients, such as preservatives, including methyl-paraben or ethyl-paraben, perfumes, dyes or the like, that are known in the art to provide desirable stability, fragrance or color, or other desirable properties, to compositions for application to the skin.

The compositions formulated as solutions or suspensions may be applied directly to the skin, or, may be formulated as an aerosol and applied to the skin as a spray, foam or mousse. The aerosol compositions further contain from about 20% to 80%, preferably from 30% to 50%, of a suitable propellant. Examples of such propellants are the chlorinated, fluorinated and chlorofluorinated lower molecular weight hydrocarbons. Nitrous oxide, carbon dioxide, butane, and propane are also used as propellant gases. These propellants are used as known in the art in a quantity and under a pressure suitable to expel the contents of the container. In the case of a pressurized aerosol, the dosage unit may be determined by providing a valve to deliver a metered amount.

In other embodiments the compositions formulated as solutions, suspensions lotions and gels of the present invention are formulated as a foam or mousse for dermal application.

### Foam and Mousse

In certain embodiments foam or mousse formulations are preferred. As used herein, the term “foam” or “mousse” is defined as comprising any lightweight material in cellular form which is made by introducing gas bubbles into liquid.

The compositions of the present invention may be altered into the form of a foam or mousse by methods known in the art for producing foam or mousse. For detailed description of such methods see for example: US Patent Application Publications 20040057922; 20020018812 and U.S. Pat. Nos. 6,730,288; 5,369,131, International Patent Application Publication No. WO 2004/037225, all incorporated herein by reference. In some embodiments the foamable composition, when placed in an aerosol container and combined with a liquefied gas propellant, releases a therapeutically beneficial foam or mousse product. In other embodiments the composition may be formulated as non-aerosol foam, for example in a propellant free dispenser.

### Shampoos and Soaps

The compositions of the present invention may further be formulated as for use as a scalp treatment, shampoo or body wash, or hair restoration or conditioning treatments. Non-limiting examples of compositions for use as a shampoo or soap may be found in U.S. Pat. No. 5,510,120 which discloses a cosmetic composition for topical application to skin and or hair comprising lectin bound particles selected from the group consisting of microcapsules and liposomes.

### Ointments

Ointments provide an effective method to apply the active agents to the skin. An ointment can be formulated with a synthetic polymer such as PEG 400 and or PEG 4000 or can be based on mineral oil and petrolatum. In certain embodiments the dermal ointment of the present invention has the following composition: about 70% to about 80% (w/w) PEG 400; about 15% to about 25% (w/w) PEG 4000; about 1% to about 15% (w/w) Steareth-20; and about 0.1 to about 1% (w/w) vitamin E.

Other dermal ointment compositions are based on petrolatum and have the following composition: about 35% to about 60% (w/w) petrolatum; about 35% to about 55% (w/w) mineral oil; about 1% to about 10% (w/w) Steareth-2; and about 0.1 to about 1% (w/w) vitamin E.

### Lotions and Creams

The compositions of the present invention may be provided as a lotion or as a cream and may include at least one or more emollient, which can function as either or both a lubricating and thickening agent. The emollients can comprise in total from about 0.1% to about 50%, preferably from about 1% to about 10%, by weight of the composition. Any emollients known to those of skill in the art as suitable for application to human skin may be used. These include, but are not limited to: hydrocarbon oils and waxes, including mineral oil, petrolatum, paraffin, ceresin, ozokerite, microcrystalline wax, polyethylene, and perhydrosqualene; silicone oils; triglyceride fats and oils, including those derived from vegetable, animal and marine source; including jojoba oil and shea butter; acetoglyceride esters, such as acetylated monoglycerides; ethoxylated glycerides, such as ethoxylated glyceryl monostearate; fatty acids, fatty alcohols and derivatives thereof. Other suitable emollients include lanolin and lanolin derivatives; polyhydric alcohols and polyether derivatives; polyhydric alcohol esters; wax esters; vegetable waxes; phospholipids, such as lecithin and derivatives; sterols, including, but not limited to, cholesterol and cholesterol fatty acid esters; amides, such as fatty acid amides, ethoxylated fatty acid amides, and solid fatty acid alkanolamides.

The lotions may further contain from about 1% to about 10%, more preferably from 2% to 5%, of an emulsifier. The emulsifiers can be nonionic, anionic or cationic. Those with skill in the art may choose emulsifiers suitable for dermal compositions.

Other conventional components of such lotions and creams may be included. One such additive is a thickening agent at a level from 1% to 10% of the composition. Examples of suitable thickening agents were disclosed for the gels, above.

The lotions and creams are formulated by simply admixing all of the components together. Preferably the components are dissolved, suspended or otherwise uniformly dispersed in the mixture.

### Solutions and Suspensions

The solutions, which may be aqueous or non-aqueous, are formulated to contain an effective concentration of an active agent.

Suitable organic materials useful as the solvent or a part of a solvent system are as follows: propylene glycol, polyethylene glycol, polypropylene glycol, glycerin, sorbitol esters, 1,2,6-hexanetriol, ethanol, isopropanol, diethyl tartrate, butanediol, and mixtures thereof. Such solvent systems can also contain water.

When the compositions of the invention are formulated as an emulsion, the proportion of the fatty phase may range from about 5% to about 80% by weight, and preferably from about 5% to about 50% by weight, relative to the total weight of the composition. Oils, emulsifiers and co-emulsifiers incorporated in the composition in emulsion form are selected from among those known to those with skill in the cosmetic or dermatological field. A gelling agent optionally may be added to the formulation. Gelling agents that are suitable for use in the present invention include, but are not limited to, cellulose ethers, alginates, polyacrylates, bentonite, gelatin, tragacanth, carbomer 940, polyvinylpyrrolidone, polyvinyl alcohol, and polyoxyethylene/polyoxypropylene block copolymers, some of which are known as poloxamers. The poloxamer compounds are sold collectively under the trademark PLURONIC (BASF, Parsippany, N.J.). PLURONIC F-127 corresponds to poloxamer 407. Other PLURONICS may be used in the present invention.

Optionally, a preservative, such as benzyl alcohol, EDTA, vitamin E tocopherol, ascorbyl palmitate, ascorbic acid, alpha lipoic acid or sorbic acid, can be added to the composition. Other preservatives well known to those of ordinary skill in the art can be used in the composition.

Agents for improving the aroma of the formulation, or fragrances, can optionally be added to the composition. A desired fragrance is honey almond oil (PCAA, Kinghurst, Houston, Tex.). Other fragrances include, but are not limited to, avocado oil, sesame oil, castor oil, olive oil, grapeseed oil, clove oil, groundnut oil, corn oil, lemon oil, coconut oil, lime oil, hazelnut oil, jojoba oil, carthamus oil and wheatgerm oil. The oils can be added individually or in combination. It is to be understood that various fragrances and assorted floral scents may be optionally added to the composition and are commercially available (PCAA, Houston, Tex.). Stabilizers, antioxidants, preservatives, humectants, regreasing agents, solvents or auxiliaries can be added to improve stability and/or adhesiveness of the formulations.

The composition of the present invention can be administered topically either once daily or several times per day, depending upon the nature and severity of the condition being treated.

It will be appreciated that other embodiments and uses will be apparent to those skilled in the art and that the invention is not limited to these specific illustrative examples.

### Example 1

### Topical Filler Composition

A table of the ingredients and their respective percentages in two embodiments (A and B) for a first aqueous composition, or topical filler, in accordance with the subject invention, is provided as Table 1, below:

TABLE 1

| | % | % |
|---|---|---|
| Ingredients | A | B |
| Water | 84.06 | 77.4505 |
| Glycerin | 10.00 | 17.75 |
| XanthanGum | 1.76 | 1.7071 |
| Caprylyl Glycol | 1.00 | 1.57 |
| Calcium Pantothenate | 0.72 | 0.1246 |
| Ethylhexylglycerin | 0.58 | 0.049 |
| Urea | 0.40 | 0.07 |
| Magnesium Lactate | 0.36 | 0.063 |
| Potassium Lactate, Water | 0.24 | 0.0252 |
| Potassium Chloride | 0.16 | 0.028 |
| Magnesium Chloride | 0.04 | 0.007 |
| Glucose | 0.04 | 0.0007 |
| Sodium Citrate | 0.03 | 0.0049 |
| Carrageenan (Chondrus Crispus) | 0.01 | 0.15 |
| Citric Acid | q.s. | — |
| Phenoxyethanol | 0.60 | 1.00 |
| TOTAL | 100.0 | 100.00 |

The listed ingredients are weighed, and admixed to provide a homogeneous mixture of the first aqueous composition.

### Example 2

### Hydration or Moisture “Trap” Composition

A table of the ingredients and their respective percentages in two embodiments (C and D) for a second lipophilic composition, also referred to as the barrier or trap composition, is provided as Table 2, below:

| | % | % |
|---|---|---|
| Ingredients | C | D |
| Cyclopentasiloxane | 64.15 | 67.47195 |
| Ethylhexyl Cocoate | 23 | 17.94 |
| Dimethiconol | 11 | 13.75195 |
| Glyceryl Linoleate | 0.90 | 0.03 |
| Glyceryl Linolenate | 0.10 | 0.03 |
| Tocopheryl Acetate | 0.31 | 0.009 |
| Ceramide | 0.029 | — |
| Lecithin | — | 0.0009 |
| Glycolipids | 0.001 | 0.0009 |
| Dimethylmethoxy Chromanol | 0.01 | 0.0003 |
| Phenoxyethanol | 0.50 | 0.765 |
| TOTAL | 100.0 | 100.00 |

The listed ingredients are weighed, and admixed to provide a homogeneous mixture of the second lipophilic composition.

### Example 3

### Application of Composition to the Human Face

A first composition and second composition can be dispensed in equal amounts, mixed together, and can be immediately applied topically to the face of a 40-50 year-old female exhibiting wrinkling of the periorbital skin at the lateral margin of the orbit. Within 1 hour, the wrinkled appearance of the skin can be dramatically diminished. This skin can appear fuller and smoother. The wrinkling of the skin can be greatly reduced and fine lines generally less evident.

The smoother, younger, and less wrinkled appearance of the skin would be maintained by once-daily, twice-daily, or three-times daily application of the first and second compositions in combination, and can visibly improve by 1-3 times daily application for a period of 10 days or more.

It should be understood that the foregoing relates only to preferred embodiments of the present invention and that numerous modifications or alterations may be made therein without departing from the spirit and scope of the invention. Although the illustrations and examples are for purposes of clarity of understanding, it will be obvious that certain changes and modifications may be practiced within the scope of the appended claims. Modifications of the above-described modes of practicing the invention that are obvious to persons of skill in the art are intended to be included within the scope of the following claims.

1. A mixture for reducing the appearance of lines, creases or wrinkles in skin, said mixture comprising:
a first aqueous composition comprising urea, lactic acid or lactate salt, and calcium pantothenate; and
a second lipophilic composition comprising one or more lipid selected from a sphingolipid, a phospholipid and a glycolipid.

2. The mixture of claim 1 wherein the urea is provided in an amount ranging from about 0.05% to about 1.0%.

3. The mixture of claim 1 wherein the lactic acid or lactate salt is provided in an amount ranging from about 0.01% to about 1.0%.

4. The mixture of claim 3 wherein the lactic acid or lactate salt is magnesium lactate in an amount from about 0.06% to about 0.6%.

5. The mixture of claim 3 wherein the lactate acid or lactate salt is potassium lactate in an amount of about 0.025%.

6. The mixture of claim 1 wherein the calcium pantothenate is provided in an amount ranging from about 0.1% to about 1.5%.

7. The mixture of claim 1 wherein the first composition further comprises a glycerin ingredient.

8. The mixture of claim 7, wherein the glycerin is provided in an amount of about 5% to about 20%.

9. The mixture of claim 7 further comprising ethylhexyl glycerin in an amount of about 0.01% to about 1.0%.

10. The mixture of claim 1 wherein the second lipophilic composition comprises one or more lipid selected from ceramide, lecithin and glycolipid.

11. The mixture of claim 10 wherein the lipid is lecithin provided in an amount from about 0.0005% to about 0.001%.

12. The mixture of claim 10 wherein lipid is lecithin and glycolipid provided in an amount ranging from about 0.001% to about 0.05%.

13. The mixture of claim 10 wherein the second lipophilic composition further comprises one or more additional ingredients selected from the group consisting of an oil, a lipid, a lubricant; a vitamin or nutrient, and a solvent.

14. The mixture of claim 1, wherein said first aqueous composition and said second lipophilic composition are contained separately or individually.

15. The mixture of claim 1 wherein said first aqueous composition and said second lipophilic composition are contained separately or individually within a single, dual-chambered container.

16. The mixture of claim 1 wherein said first aqueous composition and said second lipophilic composition are combined and contained in a single container.

17. The mixture of claim 1 wherein each of said first and second compositions are formulated for use as a gel, a lotion, a cream, a liquid solution, suspension or emulsion; a semi-solid, or a solid.

18. The mixture of claim 1 wherein at least one of said first and second compositions further comprises a fragrance or colorant.

19. A mixture of claim 1, wherein said first aqueous composition comprises ingredients and amounts shown in the table as follows:

| Ingredients | % |
|---|---|
| Water | 84.0 |
| Glycerin | 10.0 |
| XanthanGum | 1.76 |
| Caprylyl Glycol | 1.00 |
| Calcium Pantothenate | 0.72 |
| Ethylhexylglycerin | 0.58 |
| Urea | 0.40 |
| Magnesium Lactate | 0.36 |
| Potassium Lactate, Water | 0.24 |
| Potassium Chloride | 0.16 |
| Magnesium Chloride | 0.04 |
| Glucose | 0.04 |
| Sodium Citrate | 0.03 |
| Carrageenan (Chondrus Crispus) | 0.01 |
| Citric Acid | q.s. |
| Phenoxyethanol | 0.60 |
| TOTAL | 100. |

20. A mixture of claim 1, wherein said second lipophilic composition comprises ingredients and amounts shown in the table as follows:

| Ingredients | % |
|---|---|
| Cyclopentasiloxane | 64.15 |
| Ethylhexyl Cocoate | 23 |
| Dimethiconol | 11 |
| Glyceryl Linoleate | 0.90 |
| Glyceryl Linolenate | 0.10 |
| Tocopheryl Acetate | 0.31 |
| Ceramide | 0.029 |
| Glycolipids | 0.001 |
| Dimethylmethoxy Chromanol | 0.01 |
| Phenoxyethanol | 0.50 |
| TOTAL | 100.0 |

21. A mixture of claim 1, wherein said first aqueous composition comprises ingredients and amounts shown in the table as follows:

| Ingredients | % |
|---|---|
| Water | 77.4505 |
| Glycerin | 17.75 |
| XanthanGum | 1.7071 |
| Caprylyl Glycol | 1.57 |
| Calcium Pantothenate | 0.1246 |
| Ethylhexylglycerin | 0.049 |
| Urea | 0.07 |
| Magnesium Lactate | 0.063 |
| Potassium Lactate, Water | 0.0252 |
| Potassium Chloride | 0.028 |
| Magnesium Chloride | 0.007 |
| Glucose | 0.0007 |
| Sodium Citrate | 0.0049 |
| Carrageenan (Chondrus Crispus) | 0.15 |
| Citric Acid, q.s. (optional) | — |
| Phenoxyethanol | 1.00 |
| TOTAL | 100.00 |

22. A mixture of claim 1, wherein said second lipophilic composition comprises ingredients and amounts shown in the table as follows:

| Ingredients | % |
|---|---|
| Cyclopentasiloxane | 67.47195 |
| Ethylhexyl Cocoate | 17.94 |
| Dimethiconol | 13.75195 |
| Glyceryl Linoleate | 0.03 |
| Glyceryl Linolenate | 0.03 |
| Tocopheryl Acetate | 0.009 |
| Lecithin | 0.0009 |
| Glycolipids | 0.0009 |
| Dimethylmethoxy Chromanol | 0.0003 |
| Phenoxyethanol | 0.765 |
| TOTAL | 100.00 |

23. A method for reducing appearance of lines, creases, or wrinkles of skin, said method comprising the steps of:
a) providing a first aqueous composition comprising urea, lactic acid or lactate salt, and calcium pantothenate;
b) providing a second lipophilic composition comprising one or more lipid selected from sphingolipid, phospholipid and glycolipid;
c) mixing the first and second compositions together to form a substantially homogeneous mixture of the first and second compositions; and
d) applying the mixture of step (c) to the skin to rejuvenate and moisturize the skin.

24. The method of claim 23, said method comprising applying the composition two or more times per day.

25. The method of claim 23, wherein the skin is facial skin.

26. An article of manufacture comprising:
a) a first container housing a volume of a first aqueous composition comprising urea, lactic acid or lactate salt, and calcium pantothenate;
b) a second container housing a volume of a second lipophilic composition comprising one or more lipid selected from sphingolipid, phospholipid and glycolipid; and
c) written instruction for dispensing and use or application of each of said first and second compositions.

27. The article of manufacture of claim 26 wherein said first container and said second container are separate containers packaged together with said written instruction as a single, unitary package.

28. The article of manufacture according to claim 26, wherein said first container and said second container are provided as a single, dual-chambered container.

29. The article of manufacture of claim 28 wherein the first and second containers each include a dispensing nozzle associated with a respective chamber.

30. The article of manufacture of claim 28, wherein each chamber communicates with a single dispensing nozzle.

31. The article of manufacture of claim 30, wherein said single dispensing nozzle comprises a separated or partitioned nozzle opening for dispensing both compositions separately, but at the same time.
